(19) United States
(12) Patent Application Publication
(10) Pub. No.: US 2014/0271451 A1
(43) Pub. Date: Sep. 18, 2014

(54) METHOD FOR REMOVING SULFUR COMPOUNDS FROM SOUR GAS STREAMS AND HYDROGEN RICH STREAMS

(71) Applicant: TERRAVIRE, CORP., Eatontown, NJ (US)

(72) Inventors: JAMES P. BUCCINI, BASKING RIDGE, NJ (US); WOLFGANG H. KOCH, STERLING, IL (US); RAYMOND C. STENGER, FREEHOLD, NJ (US); JAMES A. WASAS, PLACENTIA, CA (US)

(73) Assignee: TERRAVIRE, CORP., EATONTOWN, NJ (US)

(21) Appl. No.: 13/800,203

(22) Filed: Mar. 13, 2013

(51) Int. Cl. C01B 3/56; C07C 7/12

(52) U.S. Cl. CPC C01B 3/56; C07C 7/12
USPC 423/658.3; 585/823; 423/419.1; 423/604; 423/473; 423/561.1; 423/557

(57) ABSTRACT

The present invention relates to a process for purifying a gas stream comprising hydrogen sulfide or mercaptans, or mixtures thereof. The gas stream can be a sour natural gas stream, a landfill gas or an industrial gas stream. The process comprises contacting the gas stream at effective absorption conditions including an absorption temperature less than about 300° C. with a solid absorbent effective to absorb the hydrogen sulfide, or mercaptans or mixtures thereof to provide a purified gas stream. Method is useful for treating gas streams having up to 90 vol-% hydrogen sulfide, or treating highly pure hydrogen streams. The invention is useful as a guard bed for fuel cells and sensitive laboratory instruments. The invention can also be employed to treat steam reformer product hydrogen streams without the need for further compression of the product hydrogen streams.

### FIELD OF THE INVENTION

This invention concerns generally with a method for removing sulfur compounds including hydrogen sulfide and mercaptans from a gas stream. More particularly, the instant invention relates to a method for reducing the concentration of sulfur compounds in a sour gas stream. Most particularly, the instant invention relates to a method and an apparatus for removing sulfur compounds from a hydrogen rich gas stream in the presence of an absorbent effective for reducing the concentration of such sulfur compounds in the hydrogen gas stream to very low levels. The method also can be employed to remove sulfur compounds from high purity hydrogen streams and sour gas streams.

### BACKGROUND OF THE INVENTION

Elemental hydrogen is generally not found in nature, because hydrogen is extremely reactive and the lightest element in the Periodic Table of Elements. Hydrogen is a desirable fuel because it is a clean burning fuel, i.e., its combustion produces only water. Hydrogen may also be used as a fuel in directly generating electricity from fuel cells. Unfortunately, pure hydrogen is very expensive to produce and very difficult to store or transport.

Typically, hydrogen is produced from natural gas or process gas streams that comprise hydrogen sulfide. In fact, many natural gas and process gas streams in addition to hydrogen sulfide, also comprise carbon dioxide, carbon monoxide, mercaptans and other impurities or contaminants. It is desirable to remove hydrogen sulfide or hydrogen sulfide and mercaptans from the natural gas before using the natural gas commercially. Combustion of hydrogen sulfide and/or mercaptans produces sulfur oxides which are toxic and when exposed to water vapor can become corrosive. When exposed to the atmosphere, sulfur oxides can result in the production of acid rain. Hydrogen sulfide and mercaptans occur naturally in natural gas. When such sulfur compounds are present in the natural gas, the natural gas is referred to as “sour gas”, particularly when the hydrogen sulfide concentration is high. Hydrogen sulfide is also produced while refining petroleum and in other processes. Natural gas may contain as much as 90% hydrogen sulfide. Hydrogen sulfide is found in hydrogen gas streams produced for hydrogen fuel cells and other applications, and is the most difficult impurity to remove from hydrogen, because both hydrogen and hydrogen sulfide are very powerful reducing agents. PEM fuel cells typically contain a solid polymer as an electrolyte and employ porous carbon electrodes containing a platinum metal catalyst. The platinum metal catalyst is easily damaged when exposed to even the smallest traces of hydrogen sulfide. Thus, the removal of as much hydrogen sulfide as possible from fuel cell hydrogen gas is extremely important to maintaining the life and the conversion efficiency of the fuel cell containing the costly platinum metal catalyst.

Typically, PEM fuel cells need only very pure hydrogen and oxygen from the air that do not comprise sulfur compounds such as hydrogen sulfide and/or mercaptans in order to operate. Typically steam reforming is employed to produce pure hydrogen gas streams for fuel cells. The reformate, or hydrogen product stream from the steam reforming process, can comprise impurities such as carbon monoxide, carbon dioxide, hydrogen sulfide, nitrogen and water. For many applications, the hydrogen product stream has to have a high hydrogen concentration, e.g., 98 volume percent hydrogen or better. Thus, not only must carbon dioxide be removed from the hydrogen product stream, but other components contained in the hydrogen product stream must be removed, including components such as hydrogen sulfide, carbon monoxide, nitrogen and water. Options for post-processing of the hydrogen product stream to further reduce impurities such as carbon monoxide include selective catalytic oxidation, separation, and methanation.

Many fuel cells operate at different levels of hydrogen consumption per pass, or hydrogen efficiencies. For example, some fuel cell arrangements demand high purity hydrogen and consume more than about 80% of the hydrogen per pass, while others consume less than about 70% of the hydrogen per pass, and do not require highly pure hydrogen. In a case which requires high purity hydrogen gas, a pressurized hydrogen product stream is passed to a separation zone comprising a pressure swing adsorption system or a palladium membrane to produce a high purity hydrogen stream (i.e., 95 to 99.999 mol-% hydrogen) and a separation waste stream comprising unrecovered hydrogen, nitrogen, and carbon oxides. A portion of the high purity hydrogen stream may be used in a hydrodesulfurization zone to remove hydrogen sulfide and the remaining portion of the high purity hydrogen stream is passed to the fuel cell zone. Anode waste gas from the fuel cell, along with the separation waste stream is passed to a burner zone for disposal.

The concentration of hydrogen sulfide and other sulfur compounds in the hydrogen product stream produced for fuel cells must be maintained at very low levels to prevent damage to the fuel cell. Removal of sulfur components subsequent to reforming does have advantages. For instance, the reforming converts essentially all species of sulfur components typically encountered in the reformer feed stream such as organosulfides, mercaptans and carbonyl sulfide to hydrogen sulfide. Thus, hydrogen sulfide removal is required to reduce the sulfur components to acceptable concentrations.

Chemisorbents such as zinc oxide are generally employed to remove hydrogen sulfide, but such chemisorbent processes are typically carried out at temperatures between about 40° C. and about 200° C. Furthermore, there are economic limits to the degree to which hydrogen sulfide can be removed to very low levels, such as less than 10 ppbv (parts per billion volume) hydrogen sulfide in a hydrogen gas stream. Generally, these chemisorbents are not regenerable or recyclable, and following their use, they are disposed of in a landfill where they slowly degenerate and release hydrogen sulfide back into the atmosphere as landfill gas.

U.S. Publication No. 2002/0023538 also discloses a process to remove hydrogen sulfide and other contaminants. This two-step process includes using a first absorbent positioned in a fluidized bed operating at a temperature of about 20-60° C. to remove at least a portion of the contaminants and using a second absorbent positioned within another fluidized bed operating at a temperature of about 100-300° C. to remove another portion of the contaminants from a gas. A conversion element, i.e., a non-thermal plasma corona reactor, is also disclosed for converting the contaminants to elemental sulfur and hydrogen at a temperature less than 400° C.

In a paper entitled, “Sulfur Removal from Reformate,” by X. Wang, et al., presented at the 225^{th} ACS National Meeting, Mar. 23-27, 2003, it was disclosed that binary oxides of copper and a second transition metal for use in on-board fuel processing. The authors noted that when treating reformate from the production of H₂ from natural gas over ZnO, that although thermodynamic equilibrium predicts that a temperature of less than 250° C. is required to reduce the H₂S concentration to less than 10 ppbv; however, they actually observed an increase concentration of H₂S as the temperature was decreased below 300° C., due to unfavorable kinetics.

Binary oxides containing copper were found to be capable of reducing the H₂S concentration from 10 ppmv to less than 50 ppbv in reformate containing 30% H₂ and 20% water when treated over the binary oxides of copper at temperatures from 200-350° C.

Membrane and pressure swing adsorption (PSA) separations can be effective for purifying the hydrogen product since they can remove nitrogen, argon, carbon dioxide, carbon monoxide and unreacted hydrocarbon-containing compounds often found in reformate. However, membrane and pressure swing adsorption systems require that the inlet or feed gases to be at elevated pressure. In large-scale steam reformers, the use of reforming temperatures that are suitable to provide reformate at pressures acceptable for PSA and membrane separation techniques is possible. However, this is not the case with smaller-scale partial oxidation/steam reforming units which must operate at lower temperatures in order to avoid expensive metallurgy and minimize capital costs. Furthermore, stand-alone hydrogen generators, where opportunities to export steam do not exist tend to reduce the economic viability of separation methods using PSA and membranes. Still further, because increased pressure decreases the efficiency of hydrogen production in partial oxidation/steam reforming processes, stand-alone and small scale reforming would typically occur at lower pressures, requiring the hydrogen product to be compressed to a pressure acceptable for PSA and membrane use. However, the additional operating and capital costs of operating small scale hydrogen compressors is generally cost prohibitive. Moreover, membrane and pressure swing adsorption systems can be particularly disadvantageous for a smaller-scale hydrogen generator due to loss of hydrogen in the rejected raffinate stream.

U.S. Publication No. 2010/0015039 discloses a pressure swing adsorption process for removal of impurities from a steam reforming process where the pressure of the reformate is elevated. Pressure swing adsorption provides a hydrogen product stream of at least about 98 volume percent hydrogen and contains less than about 10 or 20, preferably less than about 5, ppmv of carbon monoxide. Usually the pressure swing adsorption recovers at least about 60 to about 70 percent of the hydrogen contained in the reformate stream fed to the pressure swing adsorption.

Accordingly, economic and simplified processes are sought for sweetening sour gas streams such as natural gas, landfill gas, and process gases from refinery and petrochemical processes.

Methods are sought that yield a high purity hydrogen stream of suitable quality for use in fuel cells and analytical equipment which can be carried out at temperatures below 200° C.

Methods and devices are sought for removing sulfur compounds such as hydrogen sulfide and/or mercaptans from highly pure hydrogen gas at temperatures below 40° C. to provide high purity hydrogen with very low residual concentrations of sulfur compounds.

### SUMMARY OF THE INVENTION

The invention relates to a method for reducing or removing sulfur compounds such as hydrogen sulfide and mercaptans from a gas stream to provide either a sweetened gas stream or an ultra-pure hydrogen gas stream. For applications that do not require essentially complete hydrogen sulfide removal, only a portion of the incoming sour gas need be sweetened. In one embodiment, the invention is a method for sweetening a sour gas stream. In another embodiment, the invention is a process for the removal of hydrogen sulfide from a gas stream comprising hydrogen and hydrogen sulfide. Using the process of the instant invention, it is not required to separate hydrogen sulfide or mercaptans from a feed gas stream and the process can be carried out at the available system temperature and pressure of the gas stream, significantly reducing or eliminating capital and operating costs to heat or compress the gas stream. Still further, the invention is a process for the removal of hydrogen sulfide from relatively high purity hydrogen stream to provide an ultra-high purity hydrogen stream comprising less than about 10 ppbv hydrogen sulfide.

In one embodiment, the invention is a process for sweetening a sour gas stream to provide a sweetened gas stream. The process comprises contacting the sour gas stream which comprises hydrogen or methane and a sour impurity selected from the group consisting of hydrogen sulfide, mercaptans and mixtures thereof at effective absorption conditions including an absorption temperature less than or equal to 300° C. with a solid absorbent comprising a copper oxide. The solid absorbent is selective for the regerable absorption of the sour impurity to absorb at least a portion of the sour impurity to provide the sweetened gas stream which has a reduced amount of the sour impurity.

In a further embodiment, the invention is a continuous process for the sweetening of a sour gas stream comprising water, hydrocarbons, entrained particles, and hydrogen sulfide to provide a sweetened gas stream. The process comprises:

a. passing the sour gas stream to a heater-dryer to remove at least a portion of the water from the sour gas stream or to heat the sour gas stream above its dew point from to provide a dried gas stream;

b. passing the dried gas stream to a filter zone to remove at least a portion of the entrained particles to provide a filtered gas stream;

c. passing the filtered gas stream at effective absorption conditions including an absorption temperature less than about 300° C. to at least one absorption bed of a plurality of absorption beds containing a regenerable absorbent selective for the absorption of hydrogen sulfide to provide the sweetened gas stream;

d. terminating step (c) in at least one of the absorption beds and regenerating the regenerable absorbent by contacting the regenerable absorbent with a regeneration gas comprising air or oxygen at an effective regeneration temperature to desorb at least a portion of the absorbed impurity and provide a spent regeneration stream;

e. purging the at least one absorbent bed with an inert gas stream to purge the at least one absorbent bed of any residual oxygen; and,

f. resuming step (c) in the at least one absorption bed.

In a still further embodiment, the invention is a guard bed comprising a solid absorbent comprising a roasted copper compound having an average apparent bulk density of from 1.1 to about 1.6 g/cc, an interior void volume of from about 53 to about 66 percent, and an average pore volume of about 0.14 cm³/g. The roasted copper compound was prepared by roasting a natural or synthetic copper compound having an original crystal structure at a roasting temperature above about 380° C. and below about 600° C. effective to remove at least a portion of water and carbon dioxide from the copper compound while retaining at least a portion of the original crystal structure.

### BRIEF DESCRIPTION OF THE FIGURES

FIG. 1 is a schematic process flow diagram illustrating one embodiment of the present invention.

FIG. 2 is a chart illustrating a comparison of a nitrogen absorption isotherm for a roasted sample of the absorbent of the present invention and a sample of a crude, unroasted copper compound at an absorption temperature of −196° C. (−77° K).

### DETAILED DESCRIPTION OF THE INVENTION

Preferred active absorbent materials are prepared from naturally occurring or synthetic copper compounds, such as carbonates, oxychlorides, hydroxides, oxides, sulfides or sulfates of copper. Exemplary absorbent materials include, but are not limited to, mineral compounds comprising copper, such as copper carbonates as malachite Cu₂(CO₃)(OH)₂ and azurite Cu₃(CO₃)₂(OH)₂, and copper sulfates such as antlerite Cu₃((SO₄)(OH)₄, and brochantite (CuSO₄.3Cu(OH)₂) and chemicals, such as basic copper carbonate, copper hydroxide, copper oxychloride, copper sulfide and copper sulfate. Preferably, the solid absorbent of the present invention is a natural or synthetic copper compound is selected from the group consisting of copper carbonate, copper hydroxide, copper oxychloride, copper sulfide, copper sulfate and mixtures thereof. More preferably, the solid absorbent selective for the absorption of hydrogen sulfide is a copper carbonate selected from the group consisting of Cu₂(CO₃)(OH)₂, Cu₃(CO₃)₂(OH)₂, and mixtures thereof. By way of example, a synthetic copper carbonate technical grade material would have the typical analysis shown in Table 1:

TABLE 1
BASIC SYNTHETIC COPPER CARBONATE TECHNICAL
GRADE

| COMPOUND | COMPOSITION | |
|---|---|---|
| Copper | 55.0-57.0 | wt-% |
| Iron | 0.2 | wt-% |
| Chlorides | 500 | ppm-wt |
| Sulfides | 0.04 | wt-% max. |
| Lead | 70 | ppm-wt max. |
| Cadmium | 70 | ppm-wt max. |
| MEA Insolubles | 0.5 | wt-% max. |

As a further example, some naturally occurring copper carbonates such as malachite or azurite could also include other elements from Groups I, II, III, IV, V, VI, VII, and VIII the Periodic Table of Elements. For example, a typical naturally occurring copper carbonate could have the composition shown in Table 2.

TABLE 2
COMPOSITION OF TYPICAL NATURALLY
OCCURRING COPPER CARBONATES

| COMPOUND | COMPOSITION | Units |
|---|---|---|
| Chromium | 1-10 | ppmw |
| Cobalt | 10-230 | ppmw |
| Copper | 30-60 | wt-% |
| Gold | trace | ppmw |
| Iron | 0.06-0.9 | wt-% |
| Manganese | 1-1000 | ppmw |
| Nickel | 1-50 | ppmw |
| Palladium | trace | ppmw |
| Platinum | trace | ppmw |
| Rhodium | 1-10 | ppmw |
| Silver | 1-10 | ppmw |
| Titanium | 1-90 | ppmw |
| Vanadium | 1-150 | ppmw |
| Aluminum* | trace-0.5 | wt-% |
| Zinc | trace-0.3 | wt-% |

*Aluminum 4.0-5.0 wt-% when alumina is used as a binder

These mineral compounds, both synthetic and naturally occurring are considered crude copper compounds which must be subjected to an activation process in order to function as the absorbent materials of the present invention. Because the crude copper compounds are naturally occurring or synthetic, they are generally characterized as having a theoretical copper content. By “theoretical” is meant that composition which corresponds to the supposed chemical formula, for although according to a common definition: “a mineral is a naturally occurring substance of definite and uniform chemical composition with corresponding characteristic physical properties.” Nevertheless the chemical composition of many of the copper minerals does vary within limits. For this reason differing chemical formulas have been assigned to them from time to time, derived almost always from the chemical analysis alone. Therefore, all of the copper content of the absorbents of the present invention will be expressed in terms of the theoretical copper content of the crude copper compositions. Preferably, the crude copper compounds selected for activation as absorbents in the present invention will have a copper content greater than or equal to 50 wt-% of the theoretical copper content of the crude copper compound. More preferably, the crude copper compounds selected for activation as catalysts or absorbents in the present invention will have a copper content greater than or equal to 80 wt-% of the theoretical copper content of the crude copper compound. Most preferably, the crude copper compounds selected for activation as catalysts or absorbents in the present invention will have a copper content between about 90 wt-% and less than or equal to about 99.99 wt-% of the theoretical copper content of the crude copper compound.

It is well-known that copper oxide cannot be employed in the removal of hydrogen sulfide from gases comprising hydrogen or methane or mixtures thereof because the hydrogen or the methane reduces the copper oxide to copper metal and water. (See “Classic Chemistry Experiments,” compiled by Kevin Hutchings, 2000, Chapter 53, pp 132-135, published by The Royal Society of Chemistry.) The Applicant surprisingly discovered that the preparation or roasting of the crude copper compound to form a novel absorbent which retains the original crystal structure overcomes this limitation and results in an absorbent that can remove essentially all of the hydrogen sulfide and mercaptans in a gas stream which comprises hydrogen or methane or combinations thereof. Furthermore, it is not necessary to remove any carbon oxides or hydrocarbons prior to treating the gas streams with the absorbent of the current invention. Still further, the absorbent is regenerable and can be used in a process in which the absorbent bed is periodically regenerated.

The active absorbent may be disposed as a coating on a carrier, such as rings or beads, or may be particles that are not so fine as to prevent the flow of the gas through an absorbent bed. For example, the absorbent may be comprised of copper shavings with an oxidized surface. The absorbent is, preferably, placed in a chamber of such composition as to be structurally stable and resistant to attack by the gas passing through the chamber and placed above or in contact with a collector for receiving, or draining water and purified gas. Multiple stages and additional filtration may be employed as desired to assure the elimination of entrained particulates and moisture.

The absorbent of the present invention is prepared or activated by the roasting of crude naturally occurring or crude synthetic copper compounds having a fixed lattice structure comprising copper carbonate at a roasting temperature of from about 380° C. to about 800° C. to convert at least a portion of the copper compound to an oxide form to provide a prepared absorbent while retaining the original crystal structure. More preferably, the roasting temperature is from about 400° C. to about 450° C. For example, crude naturally occurring copper carbonate ore such as malachite, which is primarily copper carbonate Cu₂(CO₃)(OH)₂, undergoes thermal decomposition to CuO, H₂O and CO₂ in several stages between 250° C. and 380° C. The water and carbon dioxide is expelled into the atmosphere, leaving copper oxide. Applicant's absorbent also may be prepared by crushing the crude natural copper compound or compacting the crude synthetic or crude naturally occurring copper compound by combining or agglomerating the copper compound with a refractory material such a silica, alumina, aluminosilicate, poly-metallic siloxane and other such binder materials well-known to those skilled in the art to achieve a desired particle size. After obtaining the desired particle size the pellets are subjected to the roasting operation at a temperature of from about 380° C. to about 800° C. to provide an activated absorbent while retaining the original crystal structure. It was discovered that the crystal structure was degraded at roasting temperatures above about 800° C. More preferably, the roasting of the copper compound to convert at least a portion of the copper carbonate to copper oxide is carried out at a roasting temperature of from about 380° C. to about 600° C., and most preferably, the roasting of the copper compound is carried out at a roasting temperature of from about 400° C. to about 450° C.

The crude, unroasted absorbent of the present invention may be characterized as having an average particle size ranging from about 4 to about 100 mesh (about 4760 to about 149 microns), having a density of from about 3.6 to about 4.0, and having an average apparent bulk density of from about 1.7 to about 2.4 g/cc. Following the roasting or preparation of the crude natural or synthetic copper compound provided an absorbent which has an average apparent bulk density ranging from about 1.1 to about 1.6 g/cc and an interior void volume of from about 53 to about 66 percent, based on the absorption of water. The average pore volume of the absorbent of the present invention was about 0.14 cm³/g compared to the pore volume of the crude copper compound of 0.0012 cm³/g.

The instant invention is largely based on a chemical reaction that reduces hydrogen sulfide (H₂S) and mercaptans below detectable levels, which by typical GC analysis are typically less than about 6 ppbv. The process is capable of reducing sulfur compounds such as H₂S and mercaptans to very low concentrations for most applications over a large range of gas flow rates, pressures and temperatures. The process can accommodate inlet feed concentrations of hydrogen sulfide up to about 99.5 vol-%. The absorbent bed of the present invention can be designed to provide long on-stream times and may be regenerated or recycled. The absorbent bed and associated filter for removing entrained particles can be designed in any size, covering large industrial process applications or small laboratory gas cleaning needs. For applications that do not require essentially total removal of sulfur compounds such as hydrogen sulfide and mercaptans, only part of the incoming sour gas may be sweetened and recombined with a bypassed gas stream to provide the desired concentration of sulfur compounds. Regeneration of the sorbent following H₂S absorption results in the production of sulfur dioxide (SO₂) which may be reacted with the H₂S in the bypassed gas to produce sulfur and water. Other optional SO₂ collection or disposal methods well-known to those skilled in the art may be employed.

FIG. 1 is a schematic process flow diagram illustrating one embodiment of the present invention. Referring to FIG. 1, a sour gas stream such as a sour natural gas stream, a landfill gas stream, or a sour gas stream from a refinery process operation in line 10 is passed to a heater-dryer 101. Typically, the sour gas stream can comprise hydrogen, hydrogen sulfide, carbon oxides, C₁ to C₈ hydrocarbons, mercaptans, and water. The sour gas stream can also comprise entrained particles which must be removed to reduce the potential for plugging the absorbent beds. In the heater-dryer 101, the sour gas stream is dried to remove at least a portion of water in the sour gas stream and provide a dried gas stream in line 12 having moisture content of less than about 1000 ppmv water or heat the gas stream above its dew point. The dried gas stream is passed via line 12 to a filter zone 102 to remove at least a portion of particulates from the dried gas stream, wherein the filter zone 102 comprises a filter having a 0.5 micron filter or less, to provide a filtered gas stream in lines 13 and 14. The filtered gas stream in line 14 at effective absorption conditions is passed to at least one absorption bed 103 of a plurality of absorption beds (shown herein as absorption beds 103, 104, and 105) via lines 16, 20, and 22. Each absorption bed of the plurality of absorption beds contains a selective absorbent of the present invention. Preferably, the effective absorption conditions include an absorption pressure of from about 25 kPa (0.25 atm) to about 101,325 kPa (1000 atm), and an absorption temperature ranging from about 5° C. to about 600° C. More preferably, the effective absorption conditions include an absorption pressure of from about 50.7 kPa (0.5 atm) to about 50,663 kPa (500 atm), and an absorption temperature ranging from about 15° C. to about 450° C. Most preferably, effective absorption conditions include an absorption pressure of from about atmospheric 101.3 kPa (1 atm) to about 10,132 kPa (100 atm), and an absorption temperature ranging from about 25° C. to about 300° C. A sweetened gas stream is withdrawn from absorption bed 103 in line 18. In a similar manner, sweetened gas streams in lines 35 and 40 are withdrawn from absorption beds 104 and 105, respectively, and collected in line 42. The sweetened gas streams in line 42 are passed to a sweetened gas blending zone 107. A sweetened gas product stream in line 44 is withdrawn from the sweetened gas blending zone 107. In the sweetened gas blending zone 107, the sweetened gas is optionally blended with a portion of the filtered sour gas stream in line 13, which is passed to the sweetened gas blending zone 107 via lines 13, 26, 52, and 50. The amount of the filtered sour gas in stream 26 which is bypassed via lines 13, 26, 52 and 50 will be determined by the desired concentration of hydrogen sulfide in the sweetened gas product stream in line 44.

If required or as necessary, the absorbent can be regenerated by treating the spent absorbent comprising copper sulfide, with a regeneration stream comprising air, oxygen mixed with a non-reactive gas, or oxygen in line 32 in a conventional manner to one or more of the absorption zones (shown in FIG. 1 as line 32 to absorption zone 103, line 34 to absorption zone 104, and line 38 to absorption zone 105) to provide a spent regeneration gas stream in line 36 comprising sulfur dioxide. The spent regeneration will also comprise nitrogen, when air is employed directly or admixed with an inert gas as the regeneration stream. Line 36 represents a regeneration gas header collecting any spent regeneration gas streams from the any of the plurality of the absorption zones 103, 104, and 105 undergoing regeneration. The spent regeneration gas stream in line 36 can be passed via line 30 to sulfur dioxide collection or disposal. Optionally, a portion of the spent regeneration gas in line 36 can be passed via lines 28 and mixed with filtered gas stream from line 46 in reaction zone 106 comprising a catalytic cleanup zone wherein the spent regeneration gas and a portion of the filtered gas stream are treated to provide a reduced sulfur gas stream in line 48 and a concentrated sulfur stream in line 54. A regeneration temperature of at least 150° C. and less than or equal to about 450° C. is employed in regenerating the absorbent in the plurality of absorption beds 103, 104, and 105 to avoid damaging the structure of the absorbent. More preferably, the regeneration temperature is maintained between about 250° C. and about 400° C. Most preferably, the regeneration temperature is maintained between about 300° C. and about 400° C. The regeneration temperature will typically be controlled in the conventional manner by adjusting the flow rate and amount of oxygen, oxygen in a non-reactive gas, or air in the regeneration stream. An inert gas stream (not shown) may be used to purge the absorption bed of any residual oxygen prior to reintroducing the filtered sour gas stream to the absorption bed. Optionally, the concentrated sulfur stream in line 54 can be passed to a sulfur recovery zone (not shown). The reduced sulfur stream in line 48 can be passed to the sweetened gas blending zone 107 via lines 48 and 50 for blending into the sweetened gas product stream in line 44.

An absorption column as described hereinabove may comprise one or several beds containing absorption media in parallel or in series. Those feed tanks, filters, piping connecting flow between columns and/or beds where so connected, pumps, valving, pressure regulators, metering equipment, flow control and microprocessor equipment utilized in one embodiment are well known in construction and function to those of ordinary skill in the art.

FIG. 2 illustrates a comparison of a nitrogen absorption isotherm for a roasted sample and a crude sample copper compound at an absorption temperature of −196° C. (77° K). The BET surface area of the crude sample was 0.5 m²/g and the BET surface area of the roasted sample was 31.0. The almost nonporous crude sample showed essentially no nitrogen absorption activity compared to that of the roasted sample copper compound.

On exposure to a hydrogen stream comprising hydrogen sulfide in hydrogen and at an absorption temperature of ambient to less than about 300° C. (less than 15° C., less than 30° C., less than 40° C., less than 100° C., less than 200° C., or less than 250° C.) the hydrogen sulfide will be absorbed by the absorbent to provide a high purity hydrogen stream having a hydrogen sulfide content of less than about 10 ppbv. More preferably, the hydrogen sulfide will be absorbed by the absorbent to provide a high purity hydrogen stream having a hydrogen sulfide content of less than about 6 ppbv. Most preferably, the hydrogen sulfide will be absorbed by the absorbent to provide a high purity hydrogen stream having a hydrogen sulfide content of less than about 3 ppbv.

Absorbent Regeneration

In another embodiment of the invention, the absorbent of the present invention may be recycled (recyclable) or regenerated. By recyclable it is meant that the spent absorbent be processed in a smelting operation as a copper ore. The absorbent of the present invention may also be regenerated by contacting said solid absorbent with a regeneration gas stream comprising oxygen at an effective regeneration temperature between about 300° C. to about 600° C. Thus, the regeneration of the absorbent may be carried out by the steps of terminating the contacting of the solid absorbent with the sour gas stream and regenerating the solid absorbent by contacting the solid absorbent with a regeneration gas at an effective regeneration temperature to desorb at least a portion of the absorbed impurity and provide a spent regeneration stream. The absorbent bed containing the solid absorbent is then purged with an inert gas stream to purge the solid absorbent of any residual oxygen prior to recontacting the solid absorbent with the sour gas stream. The regeneration gas can selected from the group consisting of air, oxygen mixed with a non-reactive gas, or oxygen.

The following examples are presented to illustrate the process, system and resulting gas of the invention. These examples are intended to aid those skilled in the art in understanding the invention. The invention is, however, in no way limited thereby.

### EXAMPLES

### Example 1

### Absorption of Hydrogen Sulfide from Dry Hydrogen Gas

A series of absorption experiments were performed for the treatment of a dry hydrogen gas having a specific amount of hydrogen sulfide. The hydrogen gas standard had a certified content of hydrogen sulfide of 108 ppb (Available from Matheson Tri-Gas, Inc, Basking Ridge, N.J.) in 99.99 Hydrogen. The absorbent beds consisted of stainless steel tubes having an inside diameter of about 1 cm and a lengths of 14 cm, 22 cm and 28 cm. The 14 cm tube contained about 25 grams of the absorbent. The hydrogen gas was passed through the absorbent beds at gas flow rates ranged from 0.25 to 6 l/min. At gas flow rates less than 3 l/min, the concentration of hydrogen sulfide in the outlet stream was less than 3 ppb. When the gas rate was doubled to 6 l/min, the outlet gas contained about 24 ppb of hydrogen sulfide. Thus, for gas flow rates below 3 l/min, the hydrogen sulfide removal efficiency ranged from 98.1 to 99 percent. When the gas rate was raised to 6 l/min, the hydrogen removal efficiency was reduced to about 81 percent. The exit gas values were measured using an isotope dilution mass spectrometry (ICP-MS) method and a variation on ASTM method D4084 (wet lead acetate photometric method) which was able to achieve successful detection limits to less than 1 ppb. Table 1 presents a summary of the hydrogen sulfide removal efficiency as a function of absorbent bed length and gas flow rate.

TABLE 1
H2S Removal from High Purity, Dry Hydrogen Gas

| Media Bed | Gas Flow | H₂S in Feed, | H₂S in | Removal |
|---|---|---|---|---|
| Length, cm | Rate, l/min | ppb | Product, ppb | Efficiency, % |
| 14 | 3 | 108 | 2.1 | 98.1 |
| 22 | 3 | 108 | 1.9 | 98.3 |
| 14 | 6 | 126 | 23.9 | 81.1 |
| 14 | 1 | 126 | 2.2 | 98.3 |
| 14 | 1 | 126 | 1.8 | 98.5 |
| 14 | 0.25 | 126 | 1.7 | 98.6 |
| 28 | 0.25 | 126 | 1.3 | 99.0 |
| 28 | 0.10 | 126 | 1.2 | 99.0 |

### Example 2

### Breakthrough Analysis

A stainless steel tube having an inside diameter of about 1 cm and a length of 14 cm was filled with about 25 grams of the absorbent. The absorbent was subjected to a total gas flow of 2.1 cubic meters of dry hydrogen gas having a hydrogen sulfide level of 108 ppb before breakthrough of the hydrogen sulfide was detected. Thus, the loading of hydrogen sulfide on the absorbent at breakthrough was 340 micrograms, corresponding to about 0.53 wt-% based on the mass of the absorbent.

### Example 3

### Post Breakthrough Operation of Absorbent Bed

The experiment of Example 2 was continued at gas flow rates of 1.0 and 0.25 and the outlet concentration of the hydrogen sulfide was measured in the outlet stream. At gas rates of 1.0 and 0.25 l/min, the outlet hydrogen sulfide concentration was measured at about 2 ppb, corresponding to a hydrogen sulfide removal efficiency of about 98 percent, based on weight.

### Example 4

### Purification of High Purity Hydrogen Gas Stream to Less than 10 ppbv Hydrogen Sulfide

A cylindrical tube having an inside diameter of about 11 mm and a length of 152.4 mm was filled with 13.5 grams of the absorbent of the present invention. A Matheson Certified hydrogen sulfide standard gas (Available from Matheson Tri-Gas, Basking Ridge, N.J.) having 108 ppb H₂S in 99.99% H₂ was passed through the cylindrical tube at ambient conditions and the output gas was connected to an SRI 8610C gas chromatograph (GC) (Available from SRI Instruments, Torrance, Calif.) equipped with a high sensitivity FPD (flame photometric detector) and a capillary column. The flow-through gas flow rate was 93 cm³/min and the detection limit of the GC was approximately 6 ppb. The height of the hydrogen sulfide peak was very small and was below the limit of detection. It was concluded that the final output gas had an H₂S concentration of below 6 ppb, which was the detection limit of the GC.

### Example 5

### Removal of Hydrogen Sulfide in the Presence of Other Gases

A series of 6 borosilicate glass columns having a nominal ID of 22 mm were each filled with an average of 76.81 grams of a granular SWAPSORB-HP absorbent (Available from SWAPSOL, Eatontown, N.J.) of the present invention, wherein the absorbent had an average particle size range of from about 8 to about 50 mesh (2380 to 297 microns). The 6 borosilicate columns were connected in series such that the bottom of the first column was placed over a gas and liquid collector/separator, and the liquid free gas was in fluid communication with the top of the next column in series; and so on in this series of 6 columns. A feed gas stream comprising a mixture of 40 vol-% of a Chemically Pure hydrogen sulfide gas-99.5% H₂S (Available from Matheson Tri-Gas, Basking Ridge, N.J.) was mixed with 20 vol-% Ultra High Purity carbon dioxide; and, the balance Chemically Pure methane gas (Available from Matheson Tri-Gas, Basking Ridge, N.J.). The columns were preheated to a temperature of about 440° C. and the feed gas stream was passed to the first column in the series of the 6 columns at a flow rate of approximately 400 cc/min. It was observed that there was no detectable odor of hydrogen sulfide over a period of about 5 hours. It was concluded that the level of hydrogen sulfide was reduced to a level of about the odor threshold or less than about 4.7 ppb which is the point at which 50% of a human panel can detect the presence of hydrogen sulfide.

### Example 6

### Removal of Hydrogen Sulfide in the Presence of Air

A single borosilicate glass column having a nominal ID of 22 mm was filled with 80 grams of SWAPSORB-HP absorbent (Available from SWAPSOL, Eatontown, N.J.) of the present invention, wherein the SWAPSORB-HP absorbent had an average particle size range of from about 8 to about 17 mesh (2380 to 1095 microns). A feed gas stream comprising a mixture of 34 vol-%—of Matheson Chemically Pure hydrogen sulfide (99.5% H₂S) gas (Available from Matheson Tri-Gas, Basking Ridge, N.J.), and the balance air at ambient conditions of about 20° C. and atmospheric pressure. The temperature of the column was observed as the feed gas stream was passed into the top of the column at a flow rate of approximately 100 cc/min. The temperature of the column gradually rose to a temperature of 240° C. GC analysis of the effluent indicated that most of the oxygen was depleted and replaced with sulfur dioxide. The level of hydrogen sulfide in the effluent gas was below the limit of detection by GC analysis of approximately 6 ppb. (Note: In the above Example 6, the absorbent actually underwent an auto regeneration, wherein the absorbent took up H₂S and the hot oxygen (in the air) serves to regenerate the absorbent.)

### Example 7

### Removal of Hydrogen Sulfide in the Presence of Other Components Below 250° C.

A single borosilicate glass column having a nominal ID of 22 mm was filled with 60.4 grams of the SWAPSORB-HP absorbent (Available from SWAPSOL, Eatontown, N.J.) of the present invention, wherein the SWAPSORB-HP absorbent had an average particle size range of from about 8 to about 17 mesh (2380 to 1095 microns). A second borosilicate column having a nominal ID of 22 mm was filled with 60.8 grams of absorbent of the same type and size as in the first column. The columns were heated to a temperature of about 223° C. and a Matheson prepared feed sour gas blend comprising 40 vol-% hydrogen sulfide, 20 vol-% carbon dioxide, 36 vol-% methane, and 4 vol-% ethane (Available from Matheson Tri-Gas, Basking Ridge, N.J.) was passed to the first column at a flow rate of about 30 cc/min, and the effluent gas from the first column was passed to the second column. The effluent from the second column was analyzed by GC repeatedly over a 6 hour period. The level of hydrogen sulfide in the effluent gas was below the detectable limit by GC analysis of approximately 6 ppb.

### Example 8

### Removal of Hydrogen Sulfide from Ultra Pure Hydrogen in the Presence of Carbon Monoxide at Room Temperature

A 316 stainless steel tube having an inside diameter of about 11 mm and a length of 152.4 mm was fitted with compression fittings and filled with 18.38 grams of SWAPSORB-HP absorbent (Available from SWAPSOL, Eatontown, N.J.) of the present invention having an average particle size less than about 297 microns (50 mesh). A gas mixture of about 1 vol-% Chemically Pure hydrogen sulfide and 1 vol-% Chemically Pure carbon monoxide and the remainder Ultra High Purity hydrogen (All gasses available from Matheson Tri-Gas, Basking Ridge, N.J.) was passed through the column at ambient conditions at a feed rate of about 100 cc/min. The effluent was measured by GC analysis and the hydrogen sulfide content of the effluent was below the limit of detection of approximately 6 ppb.

### Example 9

### Removal of Hydrogen Sulfide from Ultra Pure Hydrogen in the Presence of Carbon Dioxide at Room Temperature

A 316 stainless steel column having a nominal inside diameter of about 11 mm and a length of 152.4 mm (6 inch) was fitted with compression fittings and filled with 18.56 grams of SWAPSORB-HP absorbent (Available from SWAPSOL, Eatontown, N.J.) of the present invention having an average particle size less than about 297 microns (50 mesh). A gas mixture of about 3 vol-% chemically pure hydrogen sulfide and 1 vol-% carbon dioxide and the remainder ultra pure hydrogen was passed through the column at ambient conditions at a feed rate of about 100 cc/min. The effluent was measured by GC analysis and the hydrogen sulfide content of the effluent was below the detectable limit of about 6 ppb.

### Example 10

### Removal of Mercaptan Marker from Natural Gas

A 316 stainless steel column having an inside diameter of 11 mm and a length of 152.4 mm was fitted with compression fittings and filled with 17.61 grams SWAPSORB-HP absorbent (Available from SWAPSOL, Eatontown, N.J.) of the present invention having an average particle size less than about 297 microns (50 mesh). A commercial natural gas mixture having a mercaptan marker was passed through the column at ambient conditions at a feed rate of about 100 cc/min. The effluent was determined to have no odor.

### Example 11

### Removal of Hydrogen Sulfide at Room Temperature

A single borosilicate glass column having a nominal ID of 22 mm was filled with 100.0 grams of SWAPSORB-HP absorbent (Available from SWAPSOL, Eatontown, N.J.) of the present invention, wherein the SWAPSORB-HP absorbent had an average particle size range of from about 8 to about 17 mesh (2380 to 1095 microns). A feed gas stream comprising a 100%-vol of a Matheson Chemically Pure hydrogen sulfide (Available from Matheson Tri-Gas, Basking Ridge, N.J.) having 99.5% H₂S content was passed through the column at a feed rate of about 80 cc/min and a temperature rise of about 143° C. was observed. The effluent gas from the column was measured by GC analysis and the hydrogen sulfide content of the effluent was below the limit of detection of about 6 ppb.

### Example 12

### Removal of Hydrogen Sulfide at Room Temperature

The procedure of Example 11 was repeated using a feed rate of 40 cc/min and a temperature rise of about 39° C. was observed. As in Example 11, the concentration of hydrogen sulfide in the effluent from the column was below the detectable limit by GC analysis of about 6 ppb.

### Example 13

### Removal of Hydrogen Sulfide from Sour Natural Gas (SNG)

A 316 stainless steel column having a nominal inside diameter of 11 mm and a length of 152.4 mm was fitted with compression fittings and filled with 14.11 grams of SWAPSORB-S absorbent (Available from SWAPSOL, Eatontown, N.J.) of the present invention having an average particle size less than about 297 microns (50 mesh). The absorbent in the column was saturated with Matheson Sour Natural Gas (SNG) by passing SNG through the tube until the inlet and the outlet compositions were identical. The Matheson SNG (Available from Matheson Tri-Gas, Basking Ridge, N.J.) had a composition comprising 8 vol-% hydrogen sulfide, 8 vol-% ethane, 4 vol-% carbon dioxide, and the remainder methane. The weight of the absorbent was found to have increased by about 20 wt-% following saturation after removal of water by desiccation. The saturated column was then placed in a heated oil bath and air was passed through the tube while the temperature of the oil bath gradually increased. It was observed that there was no change in the composition of the air flowing through the tube until the temperature of the oil bath reached about 150° C. At this point, sulfur dioxide was detected. Thus, regeneration of the absorbent with air required an absorbent temperature of at least about 150° C. It is believed that at temperatures above 150° C. and below 800° C., the absorbent can be regenerated without collapsing the structure of the absorbent.

### Example 14

### Removal of Hydrogen Sulfide from Continuous Flow of SNG-Sour Natural Gas

A 316 stainless steel tube having a nominal inside diameter of about 23 mm and a length of 305 mm was fitted with compression fittings and filled with 172.6 grams of SWAPSORB-S absorbent (Available from SWAPSOL, Eatontown, N.J.) of the present invention having an average particle size less than about 297 microns (50 mesh). A stream of SNG with a composition of 8 vol-% hydrogen sulfide, 8 vol-% ethane, 4 vol-% carbon dioxide and the balance methane (All gases available from Matheson Tri-Gas, Basking Ridge, N.J.) was passed through the tube at a rate of about 1.1 liters/min at ambient temperature for a period of about 24 hours. The effluent was monitored by GC analysis and throughout the experiment, the level of hydrogen sulfide in the effluent gas was less than the concentration of hydrogen sulfide contained in chemically pure methane gas (Available from Matheson Trigas, Basking Ridge, N.J.).

### Example 15

### Removal of Hydrogen Sulfide from Continuous Flow of H₂S in Air Over SWAPSORB-HP

A 316 stainless steel tube having an inside diameter of about 11 mm and a length of 152.4 mm was fitted with compression fittings and filled with 12.28 grams of SWAPSORB-HP absorbent (Available from SWAPSOL, Eatontown, N.J.) of the present invention having an average particle size less than about 297 microns (50 mesh). A gas mixture comprising 2.16 vol-% chemically pure hydrogen sulfide gas (Available from Matheson Tri-Gas, Basking Ridge, N.J.) in air was passed through the tube at ambient temperature at a rate of approximately 100 cc/min. The effluent from the tube when analyzed by GC was found to have a concentration of hydrogen sulfide below the detection limit of about 6 ppb by GC.

### Example 16

### Removal of Hydrogen Sulfide from Continuous Flow of H₂S in Air Over SWAPSORB-S

A 316 stainless steel tube having an inside diameter of 11 mm and a length of 152.4 mm was fitted with compression fittings and filled with 15.68 grams of SWAPSORB-S absorbent (Available from SWAPSOL, Eatontown, N.J.) of the present invention having an average particle size less than about 297 microns (50 mesh). A gas mixture comprising 2.16 vol-% Chemically Pure hydrogen sulfide gas (Available from Matheson Tri-Gas, Basking Ridge, N.J.) in air was passed through the tube at ambient temperature at a rate of approximately 100 cc/min. The effluent form the tube when analyzed by GC was found to have a concentration of hydrogen sulfide in the effluent gas less than the level of hydrogen sulfide contained in Chemically Pure methane gas (Available from Matheson Trigas, Basking Ridge, N.J.)

### Example 17

### Removal of Hydrogen Sulfide and Mercaptans from Continuous Flow of Sour Natural Gas Over SWAPSORB-HP

A borosilicate glass column having an inside diameter of about 22 mm was filled with 80 grams of SWAPSORB-HP absorbent (Available from SWAPSOL, Eatontown, N.J.) of the present invention having an average particle size less than about 297 microns (50 mesh). The absorbent was heated to a temperature of about 153° C. by an external heater, and a stream of crude natural gas stream containing 350 ppm H₂S, 6.11 vol-% CO₂, methane, and mercaptans at a cylinder pressure of 6474 kPa (939 psia) was passed through the column for 8 consecutive days. The effluent gas from the column was analyzed by GC and found to have a concentration of hydrogen sulfide below the limit of detection of about 6 ppb and no odor detectable to the human nose, indicating essentially complete removal of the H2S and any mercaptans initially present in the crude natural gas stream.

### Example 18

### Removal of Hydrogen Sulfide and Mercaptans from Continuous Flow of Sour Natural Gas Over SWAPSORB-HP

The procedure of Example 17 was repeated with a second sour natural gas stream containing 24,000 ppm H2S, 7.14 vol-% CO2, methane, and mercaptans at a cylinder pressure of 5516 kPa (800 psia) using the same column of Example 17 without replacing the absorbent with fresh absorbent. Again the column was heated by an external heater to a temperature of about 153° C. and the second sour natural gas stream was passed to the column for 6 consecutive days. The effluent gas from the column was analyzed by GC and found to have a concentration of hydrogen sulfide below the limit of detection of about 6 ppb and no odor detectable to the human nose indicating essentially complete removal of the H₂S and any mercaptans initially present in the second sour natural gas stream.

While the invention has been described in detail and with reference to specific embodiments thereof, it will be apparent to one skilled in the art that various changes and modifications can be made therein without departing from the spirit and scope thereof. Thus, it is intended that the invention covers the modifications and variations of this invention provided they come within the scope of the appended claims and their equivalents.

We claim:

1. A process for sweetening a sour gas stream to provide a sweetened gas stream, said process comprising contacting the sour gas stream comprising hydrogen or methane and a sour impurity selected from the group consisting of hydrogen sulfide, mercaptans and mixtures thereof at effective absorption conditions including an absorption temperature less than or equal to 300° C. with a solid absorbent comprising a copper oxide, said solid absorbent being selective for the regenerable absorption of said sour impurity to absorb at least a portion of the sour impurity to provide the sweetened gas stream having a reduced amount of the sour impurity.

2. The process of claim 1, wherein the sour gas stream is a sour natural gas stream or a landfill gas stream or an industrial gas stream or a hydrogen gas stream.

3. The process of claim 1, wherein the sour gas stream is a hydrogen stream comprising hydrogen sulfide and the sweetened gas stream comprises less than 10 ppbv hydrogen sulfide.

4. The process of claim 1, wherein the sour gas stream is a sour natural gas stream or a landfill gas stream and comprises less than about 90 vol-% hydrogen sulfide.

5. The process of claim 1, wherein the solid absorbent is a crude copper compound selected from the group consisting of copper carbonate, copper oxychloride, copper hydroxide, copper oxide, copper sulfide and copper sulfate which has been activated by roasting at a roasting temperature of from about 380° C. to about 800° C.

6. The process of claim 5, wherein the roasting temperature is from about 380° C. to about 600° C.

7. The process of claim 5, wherein the roasting temperature is from about 400° C. to about 450° C.

8. The process of claim 5, wherein the solid absorbent has an average particle size ranging from about 4760 to about 149 microns and an average apparent bulk density of from about 1.1 to about 1.6 g/cc, an interior void volume of from about 53 to about 66 percent, and an average pore volume of about 0.14 cm³/g.

9. The process of claim 1, wherein the solid absorbent is regenerable or recyclable.

10. The process of claim 1, wherein the solid absorbent is regenerated by contacting said solid absorbent with a regeneration gas stream comprising oxygen at an effective regeneration temperature between about 300° C. to about 600° C.

11. The process of claim 3, wherein the hydrogen stream comprises from 95.0 to 99.999 vol-% hydrogen and the sweetened gas stream comprises less than 6 ppbv hydrogen sulfide.

12. The process of claim 11, wherein the sweetened gas stream comprises less than 3 ppbv hydrogen sulfide.

13. The process of claim 3, wherein the absorption temperature less than or equal to 30° C.

14. The process of claim 5, wherein the solid absorbent selective for the absorption of hydrogen sulfide is a copper carbonate selected from the group consisting of Cu₂(CO₃)(OH)₂, Cu₃(CO₃)₂(OH)₂, and mixtures thereof.

15. The process of claim 14, wherein the copper carbonate has a copper content greater than or equal to 80 wt-% of the theoretical copper content of the copper carbonate.

16. The process of claim 14, wherein the copper carbonate has a copper content of between about 90 wt-% and 99.99 wt-% of the theoretical copper content of the copper carbonate.

17. The process of claim 1, wherein the effective absorption temperature ranges from ambient to less than or equal to about 300° C.

18. The process of claim 1, wherein the effective absorption temperature ranges from ambient to less than or equal to about 100° C.

19. The process of claim 1, further comprising terminating the contacting of the solid absorbent with said sour gas stream and regenerating the solid absorbent by contacting the solid absorbent with a regeneration gas at an effective regeneration temperature to desorb at least a portion of the absorbed impurity and provide a spent regeneration stream, and purging the solid absorbent with an inert gas stream to purge the solid absorbent of any residual oxygen prior to recontacting the solid absorbent with the sour gas stream.

20. The process of claim 19, wherein the regeneration gas is selected from the group consisting of air, oxygen mixed with a non-reactive gas, or oxygen.

21. A continuous process for the sweetening of a sour gas stream comprising water, hydrocarbons, entrained particles, and hydrogen sulfide to provide a sweetened gas stream, said process comprising:
a. passing the sour gas stream to a heater-dryer to remove at least a portion of the water from the sour gas stream or heat the sour gas stream above its dew point to provide a dried gas stream;
b. passing the dried gas stream to a filter zone to remove at least a portion of the entrained particles to provide a filtered gas stream;
c. passing the filtered gas stream at effective absorption conditions including an absorption temperature less than about 300° C. to at least one absorption bed of a plurality of absorption beds containing a regenerable absorbent selective for the absorption of hydrogen sulfide to provide the sweetened gas stream;
d. terminating step (c) in at least one of the absorption beds and regenerating the regenerable absorbent by contacting said regenerable absorbent with a regeneration gas comprising air or oxygen at an effective regeneration temperature to desorb at least a portion of the absorbed impurity and provide a spent regeneration stream;
e. purging the at least one absorbent bed with an inert gas stream to purge the at least one absorbent bed of any residual oxygen; and,
f. resuming step (c) in the at least one absorption bed.

22. A guard bed comprising a solid absorbent comprising a roasted copper compound having an average apparent bulk density of from 1.1 to about 1.6 g/cc, an interior void volume of from about 53 to about 66 percent, and an average pore volume of about 0.14 cm³/g, wherein said roasted copper compound was prepared by roasting a natural or synthetic copper compound having an original crystal structure at a roasting temperature above about 380° C. and below about 800° C. effective to remove at least a portion of water and carbon dioxide from the copper compound while retaining at least a portion of the original crystal structure.

23. The guard bed of claim 21, wherein the natural or synthetic copper compound has a copper content between about 90 wt-% and less than or equal to about 99.99 wt-% of the theoretical copper content of the crude copper compound.

24. The guard bed of claim 21, wherein the natural or synthetic copper compound is selected from the group consisting of copper carbonate, copper hydroxide, copper oxychloride, copper sulfide, copper sulfate and mixtures thereof.

25. The guard bed of claim 21, wherein the natural or synthetic copper compound is a copper carbonate.
